(19) United States
(12) Patent Application Publication
(10) Pub. No.: US 2024/0335661 A1
(43) Pub. Date: Oct. 10, 2024

(54) PHASE COHERENCE-BASED ANALYSIS OF BIOLOGICAL RESPONSES

(71) Applicant: Cochlear Limited, Macquarie University, NSW (AU)

(72) Inventor: Ryan Orin MELMAN, South Coogee, NSW (AU)

(21) Appl. No.: 18/293,971

(22) PCT Filed: Jul. 27, 2022
(86) PCT No.: PCT/IB2022/056932

§ 371 Date: Jan. 31, 2024

Related U.S. Application Data
(60) Provisional application No. 63/229,604, filed on Aug. 5, 2021.

(51) Int. Cl. A61N 1/36; A61N 1/05; A61N 1/372; G16H 40/63

(52) U.S. Cl. CPC A61N 1/36038; A61N 1/0541; A61N 1/37247; G16H 40/63

(57) ABSTRACT

Presented herein are techniques for detecting/determining the presence of a target biological response via a clustering of the phase angles of one or more frequencies associated with the target biological response. The techniques presented are also directed to techniques for using the target biological response detection for closed-loop control.

### BACKGROUND

### Field of the Invention

The present invention relates generally to the techniques for using phase coherence to analyze evoked biological responses.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: delivering stimulation signal sets to tissue of a recipient; recording, via an electrode configured to be implanted in the recipient, an electrophysiological trace from the tissue in response to each of a plurality of the stimulation signal sets to obtain a plurality of electrophysiological traces; converting the plurality of electrophysiological traces from a time domain to a frequency domain to obtain a number of electrophysiological frequency component sets, wherein each electrophysiological frequency component set corresponds to one of the plurality of electrophysiological traces; determining phase angles of one or more frequency components from each of a plurality of the electrophysiological frequency component sets; and determining, based on the phase angles of the one or more frequency components from the plurality of electrophysiological frequency component sets, whether the plurality of electrophysiological traces represent a target biological response.

In another aspect, a system is provided. The system comprises: at least one electrode configured to be implanted in a recipient; a recording module configured to record a plurality of sets of evoked electrophysiological signals from tissue of the recipient via the at least one electrode; and one or more processors configured to: generate a number of frequency component sets from the plurality of sets of evoked electrophysiological signals, determine phase angles of one or more frequency components from each of a plurality of the frequency component sets, and cluster the phase angles to determine whether the plurality of sets of evoked electrophysiological signals are associated with a predetermined evoked biological response.

In another aspect, one or more non-transitory computer readable storage media are provided. The one or more non-transitory computer readable storage media comprise instructions that, when executed by one or more processors, cause the one or more processors to: receive a plurality of electrophysiological traces captured from tissue of a recipient of an implantable medical device; extract phase and frequency components from a plurality of the electrophysiological traces to generate a number of frequency component sets; determine phase angles associated with a same one or more frequency components from a plurality of the frequency component sets; cluster the phase angles associated with the same one or more frequency components from the plurality of frequency component sets; and analyze the cluster of the phase angles to determine whether or not the plurality of electrophysiological traces represent a target biological response.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. TA;

FIG. 2A is a schematic diagram illustrating a nominal electrically evoked compound action potential (ECAP) response, in the time domain;

FIG. 2B is a schematic diagram illustrating the nominal electrically evoked compound action potential (ECAP) response of FIG. 2A, in the frequency domain;

FIG. 3 is a functional block diagram illustrating an evoked biological response analysis system, in accordance with certain embodiments presented herein;

FIG. 4A is a graph schematically illustrating neural contributions to an electrically evoked compound action potential (ECAP) response;

FIGS. 4B and 4C schematically illustrate that multiple neurons can be stimulated by stimulation signals delivered via a single electrode;

FIG. 5A is a polar-plot diagram illustrating phase angle coherence clustering associated with the presence of a target biological response, in accordance with embodiments presented herein;

FIG. 5B is a polar-plot diagram illustrating phase angle coherence clustering associated with the absence of a target biological response, in accordance with certain embodiments presented herein;

FIG. 6A is a polar-plot diagram illustrating phase angle coherence clustering associated the presence of a target biological response using an expected phase angle region, in accordance with certain embodiments presented herein;

FIG. 6B is a polar-plot diagram illustrating phase angle coherence clustering associated with the absence of target biological response using an expected phase angle region, in accordance with certain embodiments presented herein;

FIG. 7 is a plot illustrating measurement data and an ECAP model, in accordance with certain embodiments presented herein;

FIG. 8 is a schematic diagram illustrating an example servo function based off a logistic curve in order to determine an ECAP threshold, in accordance with certain embodiments presented herein;

FIG. 9 is a detailed flowchart of a method illustrating the use of the servo function of FIG. 8 to determine an ECAP threshold, in accordance with certain embodiments presented herein;

FIG. 10 is a graph schematically illustrating the operations shown in FIG. 9;

FIG. 11 illustrates an example vestibular stimulator system, in accordance with certain embodiments presented herein;

FIG. 12 is a flowchart of an example method, in accordance with embodiments presented herein;

FIGS. 13A, 13B, and 13C are plots illustrating use of coherence clustering in a control function, in accordance with certain embodiments presented herein; and

FIGS. 14 and 15 are flowcharts of example control methods, in accordance with certain embodiments presented herein.

### DETAILED DESCRIPTION

A number of implantable medical devices are configured to identify/detect the presence of evoked biological (physiological) responses in the body of a recipient. As used herein, evoked biological responses are electrophysiological signals generated by body tissue in response to delivered stimulation (e.g., signals induced/evoked in response to stimulation signals). The stimulation signals can be, for example electrical stimulation signals (current signals), acoustical stimulation signals, mechanical stimulation signals, etc.

It is known that, when stimulated, certain body tissue will generate specific types of evoked biological responses, referred to herein as “target evoked biological responses” or simply “target biological responses.” The presence, or absence of, a specific target biological response can be used to characterize functionality of the underlying biological processes. Target biological responses can include, for example, electrocochleography (ECoG) responses, electrically evoked compound action potential (ECAP) responses, higher evoked potentials measured from the brainstem and auditory cortex, etc. An ECAP response, in particular, is a synchronous firing of a population of electrically stimulated auditory nerve fibers.

Presented herein are techniques for detecting/determining the presence of a target biological response via a clustering of the phase angles of one or more frequencies (e.g., a fundamental frequency) associated with the target biological response. The techniques presented are also directed to techniques for using the target biological response detection for closed-loop control.

In general, the presence of a specific biological response, the absence of a specific response, a relative ratio of specific biological responses, etc. can all be used to determine information about the underlying biological process. For example, with ECochG, the absence of an Auditory Nerve Nerophonic response when there is a large clear cochlear microphonic signal is a typical sign of Neuropathy. In another example, the absence of any response to a substantial stimulus can indicate atrophy, missing structures or poor health of the underlying systems, while the presence of both components, within a specific ratio, may just indicate that the underlying biological process is functioning as expected. As such, as used herein, detecting/determining the presence of a target biological response can include detecting/determining the occurrence of one or more specific biological responses or the occurrence of portion of one or more specific biological responses, detecting/determining the absence of one or more specific biological responses or the absence of a portion of one or more specific biological responses, and/or detecting a ratio of one or more specific biological responses or portions of one or more specific biological responses.

Merely for ease of description, the techniques presented herein are primarily described with reference to a specific implantable medical device system, namely a cochlear implant system. However, it is to be appreciated that the techniques presented herein may also be partially or fully implemented by other types of implantable medical devices. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

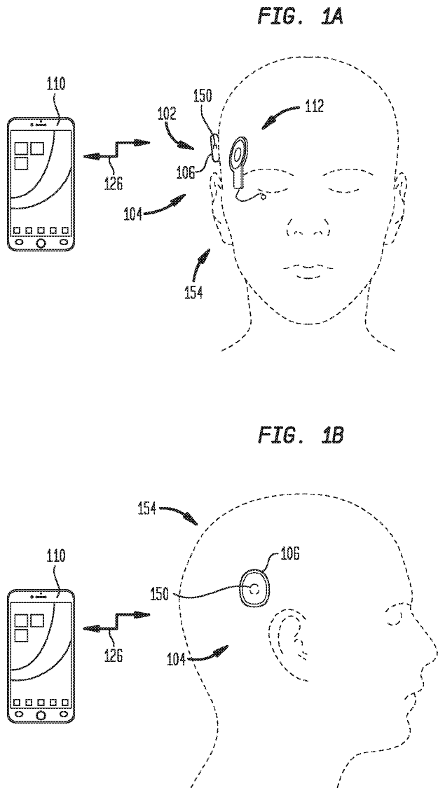

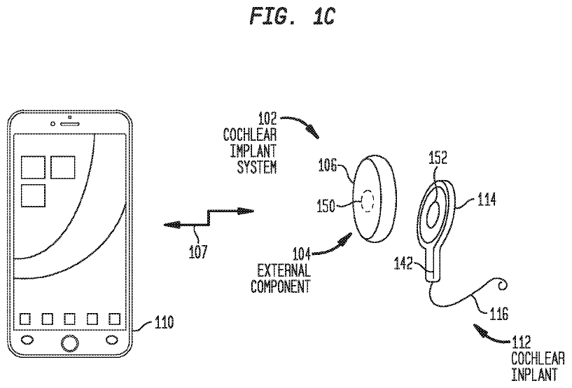

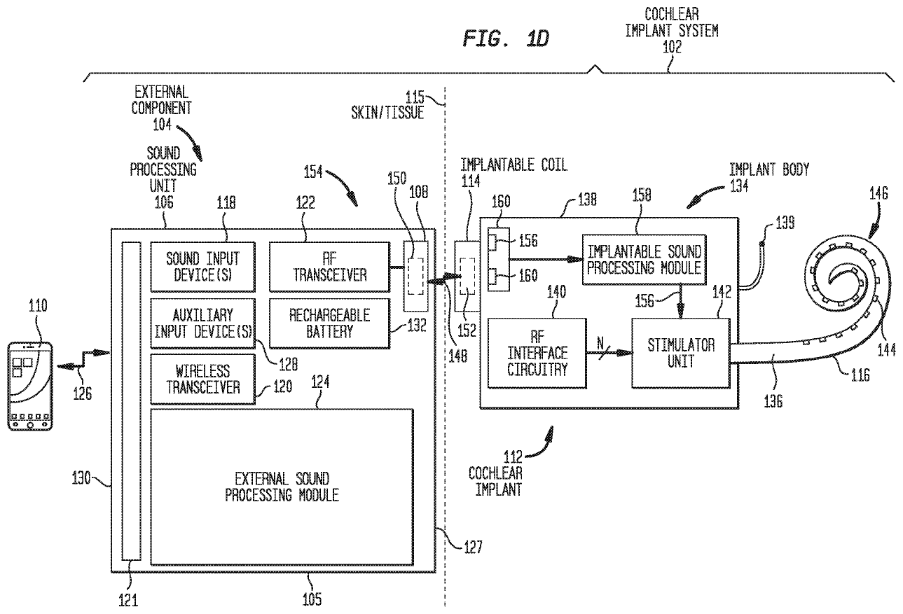

FIGS. 1A-ID illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an internal coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 110 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless RF link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

Implantable medical devices, such as cochlear implant system 112, typically rely on various evoked biological responses to determine settings/parameters for operation of the implantable medical device, where the term “evoked” is used herein in a manner synonymous with stimulation, namely an additional response (e.g., a response super-imposed on the basal response of the nerve) is created as a result of an input stimulation/stimulus. As such, there are existing techniques for determining/identifying the presence of an evoked biological response. For example, certain existing technologies for identification of Evoked Compound Action Potential (ECAP) responses identify the presence of the ECAP via a magnitude of a correlation coefficient with a template or previous measurement, coupled with a quality check of the signal to noise ratio (SNR). ECAP responses can be measured as the collective response of all neurons within a nerve or nerve portion to various stimulations, and from this the performance of the implant can be assessed and patient parameters can be interpolated. Neural response telemetry (NRT) is another term used in the art for measures of the responses of nerve cells to an evoked potential. Thus, recording an ECAP with a cochlear implant provides an objective measurement of the response of the auditory nerve to an electrical stimulus (as delivered by an implant electrode).

Although conventional measurement techniques identify the presence of evoked biological responses fairly accurately, the inventors of the present application have also identified various drawbacks with such existing techniques. In particular, in certain existing techniques, the threshold magnitude is determined by the measurement system noise floor more so than a biological process and the correlation coefficient value is affected by variations in the noise and number of samples in the measurement. This means that the noise levels and their stability have a significant impact on test/retest behavior.

Accordingly, presented herein are evoked biological response analysis techniques designed to compensate for a number of these shortcomings, especially the ability to reduce measurement time through reuse of prior measurements as well as latency shifts of a few samples, which can significantly impact the correlation co-efficient with a template even when the response morphology is correct. More specifically, in accordance with embodiments presented herein, the presence of a biological response is determined via a clustering of the phase angles of one or more frequencies associated with a specific biological response.

For ease of description, aspects of the techniques presented will be described with reference to analysis of a specific type of evoked biological response, namely an Evoked Compound Action Potential (ECAP) response. As described elsewhere herein, it is to be appreciated that the techniques can also be implemented to detect other types of evoked biological responses and can be implemented by a variety of implantable medical devices alone and/or in combination with one or more external devices.

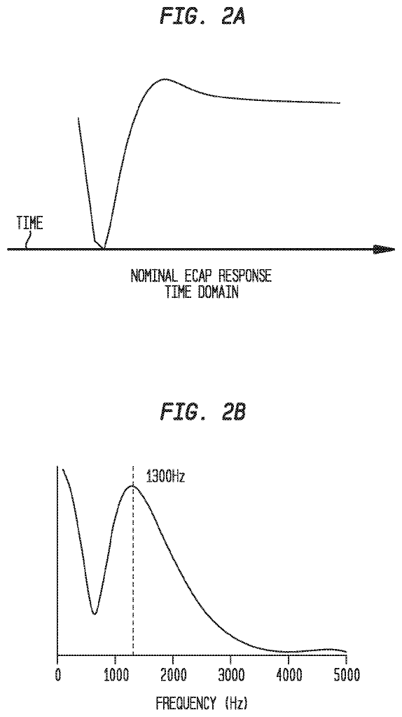

FIG. 2A is a graph illustrating a nominal/typical ECAP response captured from a cochlea of a recipient, shown in the time domain. FIG. 2B is a graph illustrating the same nominal ECAP response of FIG. 2A captured from the cochlea of the recipient, but shown in the frequency domain. The present inventors have discovered that the nominal ECAP response, in the frequency domain, is modeled by an exponential damped sinusoid, which means it has a fundamental frequency and a phase that can be tracked. The techniques presented herein make use of these properties of the nominal ECAP response to determine the presence of an ECAP response by clustering the phase angles associated one or more frequencies for each of a plurality of sets of electrophysiological signals, sometimes referred to herein as “electrophysiological traces.” If corresponding frequency portions of the plurality of electrophysiological traces have phase angles that are well clustered (e.g., the clustering exceeds a threshold), then the system determines that an ECAP response is present. However, if the corresponding frequency portions of the plurality of electrophysiological traces have phase angles that are not well clustered (e.g., the clustering is below the threshold), then the system determines that no ECAP response is present.

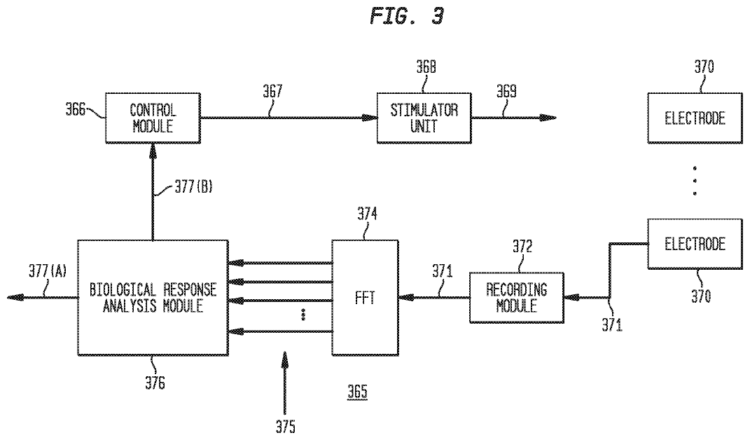

FIG. 3 is a functional block diagram illustrating an example evoked biological response analysis system 365, in accordance with certain embodiments presented herein. The evoked biological response analysis system 365 can be implemented, for example, by an implantable medical device (e.g., cochlear implant 102), by the combination of an implantable medical device and an external device (e.g., cochlear implant 102 and external device 110), multiple implantable medical devices, multiple implantable medical devices and multiple external devices, etc.

As shown, the evoked biological response analysis system 365 comprises a control module 366, a stimulator unit 368, a plurality of electrodes 370, a recording module 372, a frequency transformation module 374 (e.g., a Discrete Fourier Transform, a Goertzel Algorithm, a Least square optimization, a Matrix pencil extraction, a Wavelet transform, Recursive baysian inference (e.g. Kalman Filtering), etc.), and a biological response analysis module 376. Again, for ease of illustration, FIG. 3 will be described with reference to analysis of ECAP responses. As described elsewhere herein, it is to be appreciated that the evoked biological response analysis system 365, or a similar system, can be used to detect other types of evoked biological responses and can be implemented by a variety of implantable medical devices alone and/or in combination with one or more external devices.

In the specific illustrative ECAP example of FIG. 3, the evoked biological response analysis techniques presented herein include the capturing/recording of a plurality of sets of electrophysiological signals from tissue of the recipient. In certain examples, a medical professional (e.g., clinician, audiologist, etc.) selects one or more of the plurality electrodes 370, which are disposed within the cochlea of the recipient, for evaluation. The control module 366 provides a control signal 367 to the stimulator unit 368 that causes the stimulator unit 368 to deliver electrical stimulation signals 369 (e.g., a set of current pulses) to the recipient via, for example, the selected one or more electrodes 370 or another electrode in close proximity to the selected one or more electrodes 370. The electrical stimulation signals 369 excite auditory nerve fibers located close to the stimulating electrode and cause them to fire.

Following delivery of the electrical stimulation signals 369, an electrophysiological trace 371 (i.e., a set of electrophysiological signals representing the electrical activity from the stimulated tissue) is recorded from the tissues by the recording module 372. The recording module 372 can be configured to, among other operations, amplify the electrophysiological trace 371.

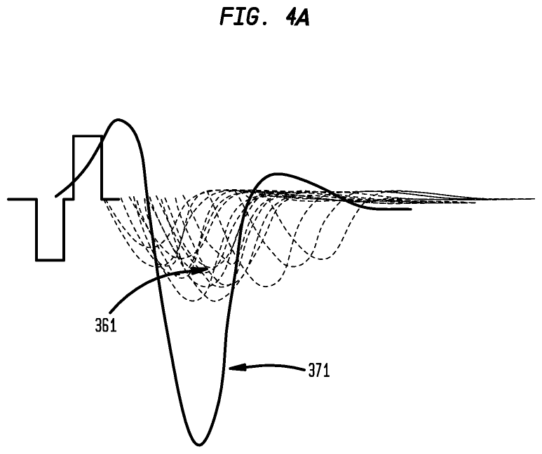

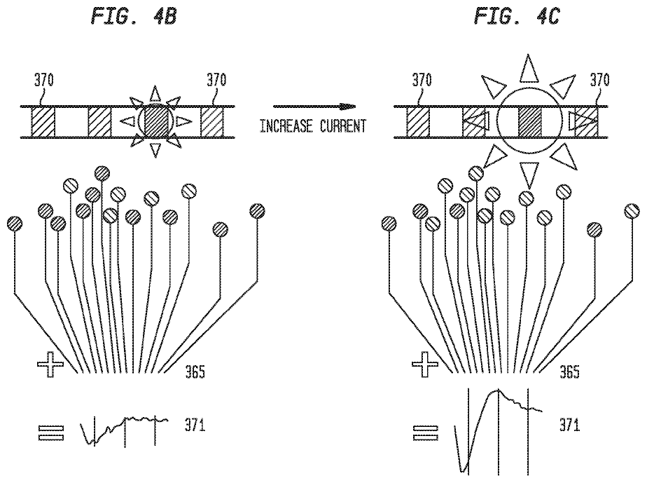

In general it is only possible to measure the electrophysiological signals when a number of neurons are stimulated together. This is shown schematically in FIGS. 4A, 4B, and 4C. In FIG. 4A, individual neuron action potentials (APs) 361, stochastically distributed, are summed to form the electrophysiological signal trace (compound action potential) 371. In FIGS. 4B and 4C, the auditory nerve 365 is shown schematically as a bundle of neurons. The plurality of electrodes 370 is situated so that stimulating current can be delivered to the nerve. As shown in FIG. 4C, increasing the current delivered via an electrode increases the number of neurons that are stimulated, and causes a corresponding increase in the electrophysiological signal trace 371.

During a typical electrophysiological measurement, stimulus is delivered over a window of time, referred to as an epoch. A “probe epoch” is one associated with a stimulus intended to evoke a biological response, in which case it is possible to start the recording before the stimulus, or an arbitrary period after it is complete (e.g., wait 100 μs). A probe stimulus doesn't have to arise from a single pulse: the probe can comprise a complex stimulus, such as from a pulse train (a series of pulses at a certain rate), or it can be multipolar, such as delivered from multiple electrodes. It is preferable to have at least 1 epoch per stimulation. In ECOG, say, it can be preferable to have more than one recording window after one stimulus because there are different types of responses at different times after the stimulus.

Returning to FIG. 3, the electrophysiological trace 371 is, when captured via the recording module 372, in the domain. The electrophysiological trace 371 is provided to the frequency transformation module 374, where the electrophysiological trace 371 is converted into the frequency domain. As shown, the frequency transformation module 374 separates the electrophysiological trace 371 into a plurality of frequency components (frequency bins), referred to herein as electrophysiological frequency components 375. The electrophysiological frequency components 375 are provided to the biological response analysis module 376.

The above process of delivering stimulation signals to the recipient and recording of a resulting electrophysiological trace 371 is iteratively repeated a number of times for the same combination of stimulation and recording electrode. For each electrophysiological trace 371, the associated electrophysiological frequency components 375 are provided to the biological response analysis module 376. That is, the biological response analysis module 376 receives/collects a plurality of different sets of electrophysiological frequency components 375, where each set of electrophysiological frequency components 375 is obtained/evoked in response to a corresponding set of one or stimulation signals (e.g., there is a one-to-one correspondence between stimulation signals and a set of electrophysiological frequency components 375).

In operation, the biological response analysis module 376 can receive and store all of the electrophysiological frequency components 375 within a given set, or only one or more specific frequency components within a given set (e.g., only the electrophysiological frequency components associated with the fundamental frequency of the electrophysiological trace). As such, the term “set of electrophysiological frequency components” can include a single frequency component, all of frequency components, or a subset of two or more frequency components.

The biological response analysis module 376 is configured use the sets of electrophysiological frequency components 375 associated with (obtained from) a plurality of electrophysiological traces 371 to determine whether or not the electrophysiological traces are ECAP responses (e.g., target responses). In accordance with embodiments presented herein, the biological response analysis module 376 is configured to determine whether or not the electrophysiological traces are ECAP responses by clustering the phase angles associated with one or more of the frequency components across the sets of electrophysiological frequency components 375 sets of electrophysiological signals.

More specifically, the biological response analysis module 376 is configured to determine the phase angle for a selected frequency component from each of a plurality of the sets of electrophysiological frequency components 375. If the determined phase angles are well clustered (e.g., the clustering exceeds a threshold), then the biological response analysis module 376 determines that the electrophysiological traces 371 are ECAP responses (e.g., an ECAP response is present). However, if the determined phase angles are not well clustered (e.g., the clustering is below the threshold), then the biological response analysis module 376 determines that the electrophysiological traces 371 are not ECAP responses (e.g., no ECAP response is present).

In certain embodiments, the biological response analysis module 376 is configured to use only the phase angles associated with a fundamental frequency of the target biological response (e.g., the nominal ECAP response) in the clustering process to determine whether or not a target biological response is present. In other embodiments, the biological response analysis module 376 is configured to use phase angles associated with multiple different frequencies of the target biological response to determine whether or not a target biological response is present.

The biological response analysis module 376 can generate one or more outputs 377 representative of the final classification/determination of whether or not an ECAP response is present. In certain embodiments, the one or more outputs can be audible or visible outputs, represented by arrow 377(A). In other embodiments, as described further below, the one or more outputs can be control outputs, represented by arrow 377(B), that are sent to, for example, the control module 366 for use as part of a closed-loop control system.

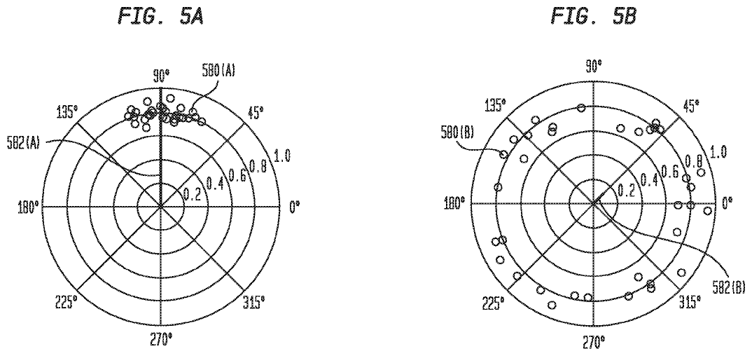

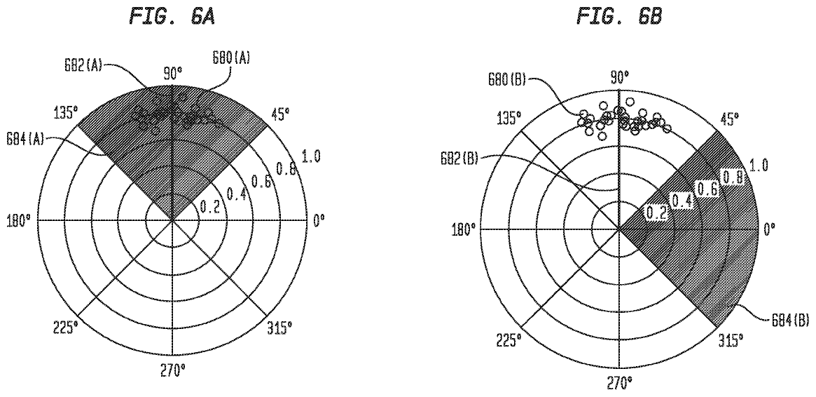

In accordance with certain embodiments presented herein, the clustering threshold (threshold for detection of a target biological response) can be based a concentration or coherence of unimodal clustering. FIG. 5A is a polar plot diagram illustrating clustering, using a unimodal clustering of phase angles, that results in the determination that an ECAP response is present, in accordance certain embodiments presented herein. In FIG. 5A, each dot 580(A) represents a determined phase angle of the same selected frequency component across a plurality of the sets of electrophysiological frequency components 375 (e.g., associated with a plurality of electrophysiological traces 371) each captured in response to the same stimulation signal(s). The vector 582(A) is has a length representing the coherence of the phase angles. A vector having a length that is greater than a predetermined threshold (e.g., coherence above a threshold) indicates that the phase angles are sufficiently clustered such that an ECAP response is present. In this example, the length of the vector 582(A) is greater than the predetermined threshold, meaning the associated electrophysiological traces 371 are ECAP responses.

While FIG. 5A represents sufficient clustering (coherence and/or concentration) to indicate presence of an ECAP response, FIG. 5B is a radar plot diagram illustrating a uniform dispersion of phase angles, that results in the determination that an ECAP response is absence, in accordance with certain embodiments presented herein. In FIG. 5B, each dot 580(B) represents a determined phase angle, and magnitude of the same selected frequency component across a plurality of the sets of electrophysiological frequency components 375 (e.g., associated with a plurality of electrophysiological traces 371) each captured in response to the same stimulation signal(s). The vector 582(B) is has a length representing the coherence of the phase angles. A vector having a length that is greater than a predetermined threshold indicates that the phase angles are sufficiently clustered such that an ECAP response is present. In this example, the length of the vector 582(B) is less than the predetermined threshold, meaning the associated electrophysiological traces 371 are not ECAP responses (e.g., coherence is below a threshold).

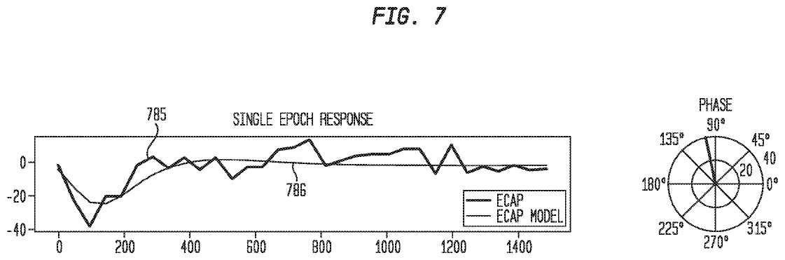

As noted, FIGS. 5A and 5B illustrate examples in which the determination of whether an ECAP response is present is based on a clustering threshold (threshold for detection of a target biological response) that, in turn, is based solely on a concentration of unimodal clustering. In accordance with further embodiments, the determination of whether an ECAP response is present can be based on clustering threshold (e.g., unimodal clustering) and a secondary determination of whether a mean phase angle is located/positioned within an “expected phase angle region,” which is a phase angle region that is correlated with the phase angles which produce plausible morphologies associated with a target biological response. Such examples are shown in FIGS. 6A and 6B.

More specifically, FIG. 6A is a polar plot diagram illustrating clustering/coherence, using a unimodal clustering of phase angles, that results in the determination that an ECAP response is present, in accordance certain embodiments presented herein. In FIG. 6A, each dot 680(A) represents a determined phase angle of the same selected frequency component across a plurality of the sets of electrophysiological frequency components 375 (e.g., associated with a plurality of electrophysiological traces 371) each captured in response to the same stimulation signal(s). The vector 682(A) is has a length representing the coherence/clustering of the phase angles. A vector having a length that is greater than a predetermined threshold indicates that the phase angles are sufficiently clustered such that an ECAP response is present. In this example, the length of the vector 682(A) is greater than the predetermined threshold, meaning the associated electrophysiological traces 371 are initially classified as ECAP responses.

In addition to the unimodal clustering, FIG. 6A also illustrates the use of an expected phase angle region 684(A). In this example, information about the nominal ECAP response is used to determine an angular region into which the phase angle, for a selected frequency component, should fall. As shown in FIG. 6A, the vector 682(A) points into the expected phase angle region 684(A). As such, the phase angles are clustered in to the expected phase angle region 684(A), confirming that an ECAP response is present (e.g., the electrophysiological traces 371 are finally classified as ECAP responses).

While FIG. 6A represents sufficient clustering/coherence to indicate presence of an ECAP response, FIG. 6B is a polar plot diagram illustrating clustering/coherence, using a unimodal clustering of phase angles, that results in the determination that an ECAP response is absence, in accordance with certain embodiments presented herein. In FIG. 6B, each dot 680(B) represents a determined phase angle of the same selected frequency component across a plurality of the sets of electrophysiological frequency components 375 (e.g., associated with a plurality of electrophysiological traces 371) each captured in response to the same stimulation signal(s). The vector 682(B) is has a length representing the coherence/clustering of the phase angles. A vector having a length that is greater than a predetermined threshold indicates that the phase angles are sufficiently clustered such that an ECAP response is present. In this example, the length of the vector 682(B) is greater than the predetermined threshold, meaning the associated electrophysiological traces 371 are initially classified as ECAP responses.

However, in addition to the unimodal clustering, FIG. 6B also illustrates the use of an expected phase angle region 684(B). In this example, information about the nominal ECAP response is used to determine an angular region into which the phase angle, for a selected frequency component, should fall. As shown in FIG. 6B, the vector 682(B) points outside of the expected phase angle region 684(B). As such, the phase angles are not clustered in to the expected phase angle region 684(B), meaning that the observed clustering is likely due to systematic artifact and an ECAP response is absent (e.g., the electrophysiological traces 371 are finally classified as not being ECAP responses).

Additionally, coherence can be used to create a measurement family for samples acquired at a specific current level to, for example, improve robustness in other direction applications. For example, the mean angle size of a concentration cluster which appear in the presence of a response can be used as a dynamic template to indicate when an acquired measurement is substantially different from existing measurements at the specified stimulation level. For example in FIG. 5A, if a single measurement is taken with a phase angle of 270 degrees, when the mean phase angle is 90 degrees and coherence is greater than 0.8, then it is likely that the most recent measurement is “out of family” and should not be used in any decision making step in the current algorithm. Families can be constructed for each fundamental frequency of the biological response and out of family specification can require a (optionally weighted) minimum family error for a measurement to be excluded. If the shift in phase is small or persistent, and families are generated with a forgetting factor, then over time the definition of the measurement family will shift making measurements which would have previously been excluded as out of family, now in family and included in the decision making paradigm of a real time control loop algorithm. This can lead to a more robust implementation of an existing controller such as a beam former which is trying to decide on the angle of incidence to enhance for speech or best location of the null for noise.

In the clustering portion of FIGS. 5A-5B and 6A-6B, the concentration or coherence/clustering for determination of whether an ECAP response is present can be set in a number of manners, such as for statistical robustness. For example, the probability of 16 independent responses having a coherence ≥0.966 is less than 1/1000000, if the underlying data is uniform and random. This correlates to a von Mises concentration (κ) greater than 15. As such the robustness of a system can be estimated using standard circular statics through integration of these parameters into the detection function. This is beneficial as the measurement noise which is out of band from the phase estimation function has a much lower impact on the phase coherence than incidental spikes or high frequency noise may have on the magnitude of the correlation coefficients in the time domain, increasing the tests robustness to noise.

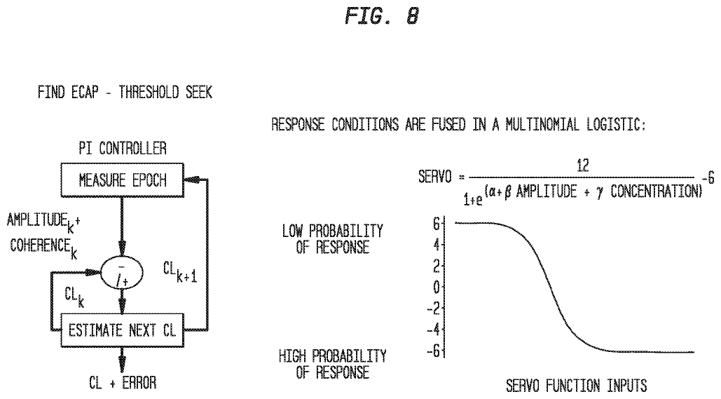

Moreover, these statistical tests can be directly paired with biologically significant measurements such as the response amplitude to provide a robust estimator of a meaningful threshold, where coherence or concentration can be used to provide evidence that the signal is a target (meaningful) biological response, and the amplitude can be utilized to find a biologically appropriate threshold. Provided the target amplitude is substantially above the noise floor of the measurement system, variations in noise floor will have little to no impact on threshold magnitude provided the noise floor remains below threshold. The noise floor can be assessed using existing techniques or by analysis of the residuals after fitting a nominal biological response to the measurement, as shown in FIG. 7 plot, where the line 785 shows the measurement data and the 786 line shows the ECAP model.

In accordance with certain embodiments presented herein, the phase angle clustering/concentration may be combined with response magnitude estimation and/or other concentration measurements at different frequencies in a “servo function” to create a closed-loop estimation controller for estimating magnitude. FIG. 8 illustrates an example servo function based off a logistic curve in order to determine an ECAP threshold. That is, FIG. 8 illustrates use of a servo function and closed loop controller for estimating a threshold. The servo drives estimation of the required stimulation current to achieve a zero value. In general, a servo function in accordance with embodiments presented is linear around the zero mark so a proportional change can be introduced to achieve convergence when in the normal operating point of the servo, and is configured to restrict/clamp the output (e.g., current change level) at the extremes to limit the rate of stimulation level change.

In accordance with certain examples, factors may be combined as per the multinomial logistic shown in FIG. 8, in the event that they are boosting confidence in the response and confidence is only one of the parameters to have high confidence in the presence of a biological response. Factors may also be combined in parallel as per Equation 1, below, which drives the stimulation level up when confidence is low for any component (e.g., Parallel servo implementation where 0<servoₖ<1)

$${Servo}_{parallel} = scale * ( \frac{0.5}{n} - {\sum }_{1}^{n} \frac{1}{{servo}_{k}} ) \qquad Equation 1$$

In accordance with certain examples, factors may be combined as per the multinomial logistic shown in FIG. 8, in the event that they are boosting confidence in the response and confidence is only one of the parameters to have high confidence in the presence of a biological response. Factors may also be combined in parallel as per Equation 1, below, which drives the stimulation level up when confidence is low for any component (e.g., Parallel servo implementation where 0<servoₖ<1)

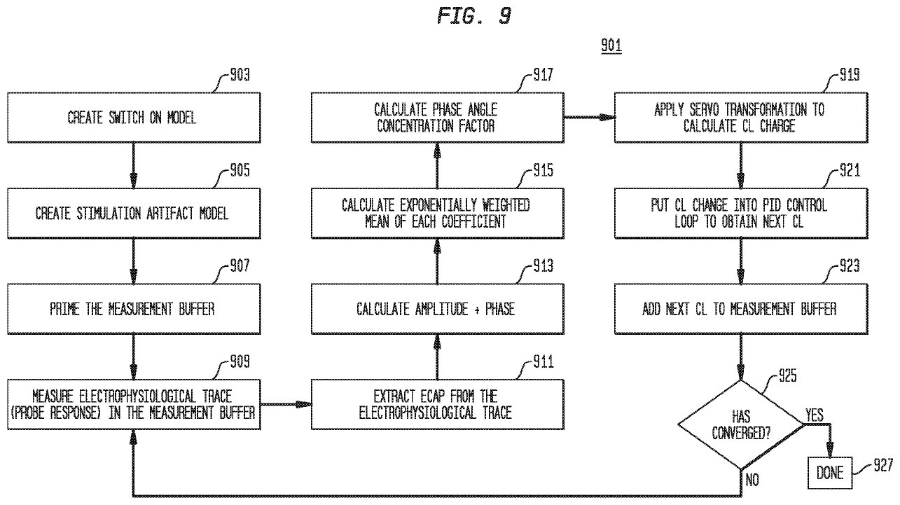

FIG. 9 is a detailed flowchart of a method 901 illustrating the use of the servo function of FIG. 8 to determine a threshold level. For ease of illustration, the method 901 will be described with reference to evoked biological response analysis system 365, used for closed-loop control (e.g., biological response analysis module 376 generating control output signals 377(B)).

Method 901 begins at 903 where a switch on model is created. At 905, an artifact model is created and, at 907, a measurement buffer is primed. At 909, the evoked biological response analysis system 365 measures/captures an electrophysiological trace (probe response) in the measurement buffer.

At 911, the biological response analysis module 376 extracts an ECAP from the electrophysiological trace and calculates the associated amplitude and phase at 913. At 915, the biological response analysis module 376 calculates an exponentially weighted mean of each coefficient and, at 917, calculates the phase angle concentration factor. At 919, the biological response analysis module 376 applies the servo function (servo transformation) to calculate current level (CL) change. At 921, the biological response analysis module 376 issues the one or more outputs 377(B) to place the current level change into the control loop and, as such, obtain the next current level. At 923, the next current level is added to the measurement buffer and, at 925, a determination is made as to whether convergence has been obtained. If not, then method 901 returns to 909 and steps 911-925 are repeated. If convergence has been obtained, then the method 901 ends at 927.

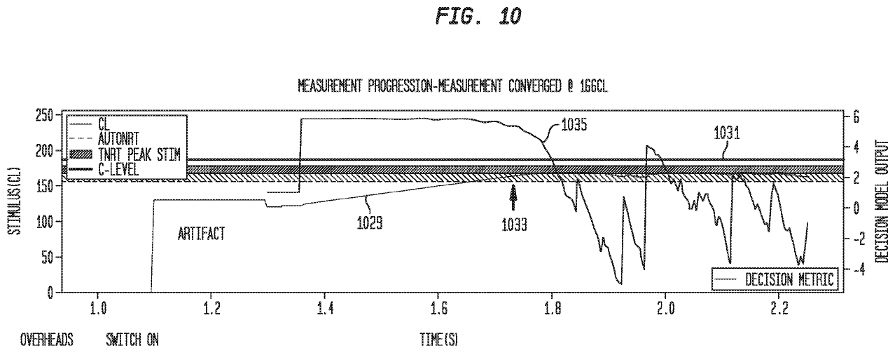

FIG. 10 is a graph schematically illustrating the operations shown in FIG. 9. In FIG. 10, line 1029 is the peak stimulation current (current level), line 1031 is the behavioral comfort level, region 1033 is the AutoNRT TNRT levels, and line 1035 is the servo function output.

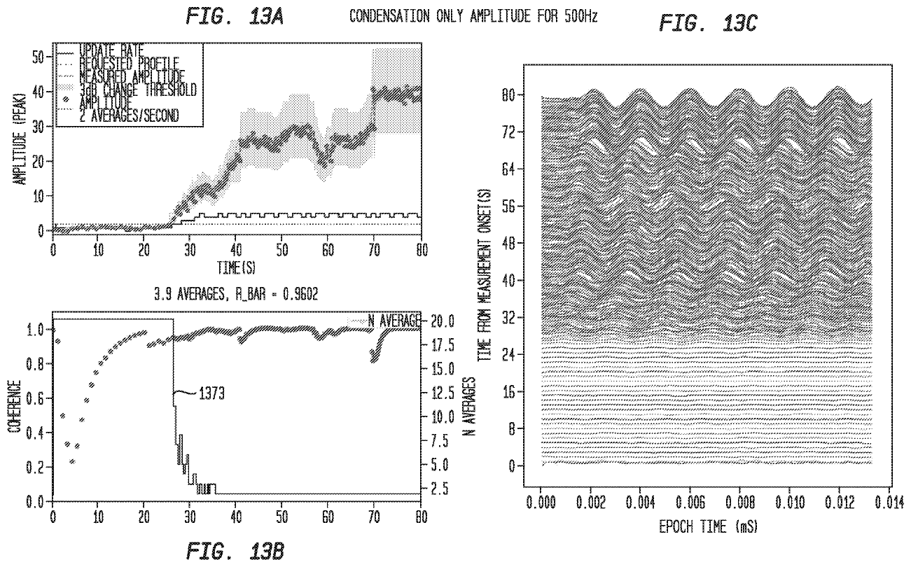

In general, FIGS. 8, 9, and 10 illustrate an example type of closed-loop control in which an input is controllably adjusted to obtain a desired output. In these examples, the input is the current level and the output is the ECAP threshold (e.g., using concentration and magnitude to control input current to get a desired amplitude). However, it is to be appreciated that this combination of input current and output amplitude is merely one example of inputs/outputs that can be controlled via a closed-loop control system, in accordance with embodiments herein. For example, the techniques presented herein could alternatively be used to control the number of samples or averages that are measured by the system (e.g., no changing of stimulation levels, but instead adapt averaging). Such embodiments are generally shown in FIGS. 13A, 13B, 13C, 14, and 15.

More specifically, FIGS. 13A, 13B, and 13C are plots illustrating the use of phase coherence to determine the number of averages to acquire. FIGS. 13A, 13B, and 13C illustrate a continuous measurement where the number of averages is inversely proportional to the coherence. In FIG. 13A, the dots represent the ECoG amplitude over time, while the shaded region represents a 3 dB change in amplitude. The line at the bottom is the rate of displayed waveform updates, where fewer averages equals a greater update rate.

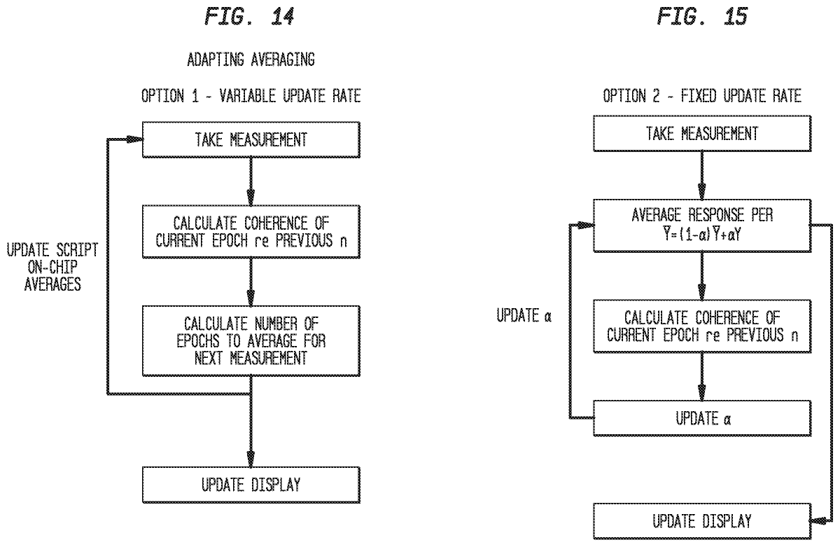

In FIG. 13B, the dots represent the phase coherence estimation and the line/trace 1373 represents the number of average epochs acquired based on the phase coherence. FIG. 13C is a cascade of averaged measurement epochs. FIGS. 14 and 15 are flowcharts illustrating two example methods for adaptive averaging, where FIG. 14 uses a variable update rate and FIG. 15 uses a fixed update rate.

Again, it is to be appreciated that FIGS. 13A-13C, 14, and 15 are merely illustrative of example implementations of the techniques presented herein and that other implementations are possible. For example, in another embodiment, a spinal cord or vagal stimulator may experience a change in electrode location relative to the target nerve with changes in posture. In such embodiments, the techniques presented herein can be to moderate the stimulation current level based on the evoked response to ensure the stimulation level is sufficient, but not too large (e.g., modulate the current delivered based on response coherence).

In yet a still other example, the techniques presented herein can be used to determine the required amplitude of acoustic/electrical stimulation of a tinnitus masker. For example, the amplitude of acoustic/electrical stimulation may be assessed as cortical processing will entrain to the stimulation if the subject is paying attention to it, which may be a sign the masker is too loud or sufficiently loud.

As noted elsewhere herein, the arrangements shown in FIGS. 1-10 are merely illustrative and that techniques presented herein may be implemented with different arrangements. Also as noted elsewhere herein, embodiments presented herein have been primarily described with reference to an example implantable medical device, namely a cochlear implant system. However, as noted above, it is to be appreciated that the techniques presented herein may be implemented by a variety of other types of implantable medical devices (or systems that include other types of implantable medical devices). For example, the techniques presented herein may be implemented by other auditory prostheses, such as acoustic hearing aids, middle ear auditory prostheses, bone conduction devices, direct acoustic stimulators, electro-acoustic prostheses, other electrically simulating auditory prostheses (e.g., auditory brain stimulators), etc. The techniques presented herein may also be implemented by tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

FIG. 11 illustrates an example vestibular stimulator system 1102, with which embodiments presented herein can be implemented. As shown, the vestibular stimulator system 1102 comprises an implantable component (vestibular stimulator) 1112 and an external device/component 1104 (e.g., external processing device, battery charger, remote control, etc.). The external device 1104 comprises a transceiver unit 1160. As such, the external device 1104 is configured to transfer data (and potentially power) to the vestibular stimulator 1112,

The vestibular stimulator 1112 comprises an implant body (main module) 1134, a lead region 1136, and a stimulating assembly 1116, all configured to be implanted under the skin/tissue (tissue) 1115 of the recipient. The implant body 1134 generally comprises a hermetically-sealed housing 1138 in which RF interface circuitry, one or more rechargeable batteries, one or more processors, and a stimulator unit are disposed. The implant body 134 also includes an internal/implantable coil 1114 that is generally external to the housing 1138, but which is connected to the transceiver via a hermetic feedthrough (not shown).

The stimulating assembly 1116 comprises a plurality of electrodes 1144 disposed in a carrier member (e.g., a flexible silicone body). In this specific example, the stimulating assembly 1116 comprises three (3) stimulation electrodes, referred to as stimulation electrodes 1144(1), 1144(2), and 1144(3). The stimulation electrodes 1144(1), 1144(2), and 1144(3) function as an electrical interface for delivery of electrical stimulation signals to the recipient's vestibular system.

The stimulating assembly 1116 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 1112, the external device 1104, and/or another external device, can be configured to implement the techniques presented herein. That is, the vestibular stimulator 1112, possibly in combination with the external device 1104 and/or another external device, can include an evoked biological response analysis system, as described elsewhere herein.

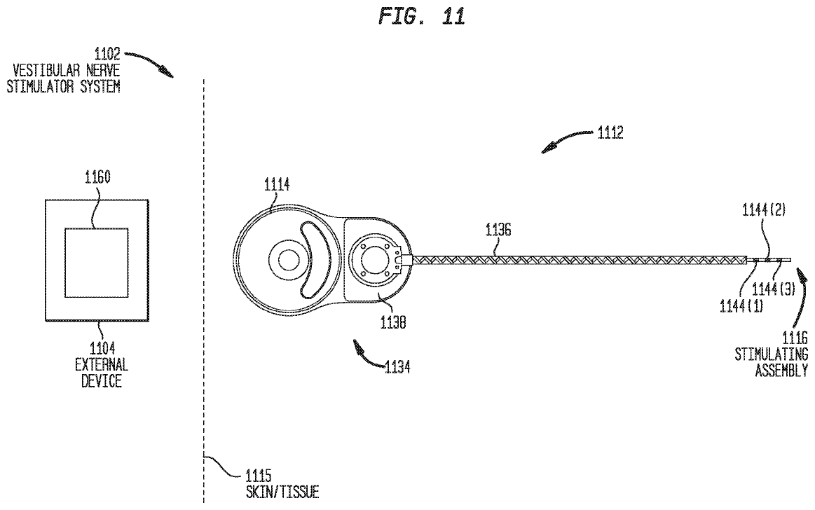

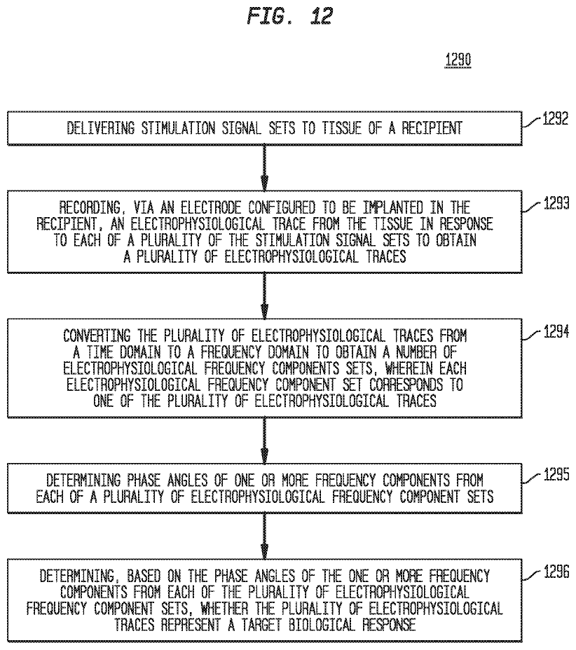

FIG. 12 is a flowchart of an example method 1290, in accordance with certain embodiments presented herein. Method 1290 begins at 1292 where stimulation signal sets are delivered to tissue of a recipient. At 1293, an implantable component records, via an electrode configured to be implanted in the recipient, an electrophysiological trace from the tissue in response to each of a plurality of the stimulation signal sets to obtain a plurality of electrophysiological traces. At 1294, one or more processors convert the plurality of electrophysiological traces from a time domain to a frequency domain to obtain a number of electrophysiological frequency component sets, wherein each electrophysiological frequency component set corresponds to one of the plurality of electrophysiological traces. At 1295, the one or more processors determine phase angles of one or more frequency components from each of a plurality of the electrophysiological frequency component sets. At 1296, the one or more processors determine, based on the phase angles of the one or more frequency components from each of the plurality of electrophysiological frequency component sets, whether the plurality of electrophysiological traces represent a target biological response.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
delivering stimulation signal sets to tissue of a recipient;
recording, via an electrode configured to be implanted in the recipient, an electrophysiological trace from the tissue in response to each of a plurality of the stimulation signal sets to obtain a plurality of electrophysiological traces;
converting the plurality of electrophysiological traces from a time domain to a frequency domain to obtain a number of electrophysiological frequency component sets, wherein each electrophysiological frequency component set corresponds to one of the plurality of electrophysiological traces;
determining phase angles of one or more frequency components from each of a plurality of the electrophysiological frequency component sets; and
determining, based on the phase angles of the one or more frequency components from the plurality of electrophysiological frequency component sets, whether the plurality of electrophysiological traces represent a target biological response.

2. The method of claim 1, wherein delivering each of the stimulation signal sets to the tissue of the recipient, comprises:
delivering one or more electrical stimulation signals to the tissue of the recipient via one or more electrodes configured to be implanted in the recipient.

3. The method of claim 1, wherein delivering each of the stimulation signal sets to the tissue of the recipient, comprises:
delivering one or more acoustical stimulation signals to the tissue of the recipient.

4. The method of claim 1, wherein delivering each of the stimulation signal sets to the tissue of the recipient, comprises:
delivering one or more mechanical stimulation signals to the recipient.

5. The method of claim 1, wherein recording an electrophysiological trace from the tissue in response to a stimulation signal set comprises:
recording a plurality of neuron action potentials for a period of time following delivery of the stimulation signal set to the tissue of the recipient.

6. The method of claim 1, wherein converting the plurality of electrophysiological traces from the time domain to the frequency domain comprises:
extracting phase and frequency components from each of the plurality of electrophysiological traces.

7. The method of claim 1, wherein determining phase angles of one or more frequency components from each of the plurality of electrophysiological frequency component sets comprises:
determining phase angles of a frequency component associated with a fundamental frequency of the target biological response.

8. The method of claim 1, wherein determining phase angles of one or more frequency components from each of the plurality of electrophysiological frequency component sets comprises:
determining phase angles of a plurality of frequency components from each of the plurality of electrophysiological frequency component sets.

9. The method of claim 1, wherein determining, based on the phase angles of the one or more frequency components from each of the plurality of electrophysiological frequency component sets, whether the plurality of electrophysiological traces represent the target biological response comprises:
clustering the phase angles of the one or more frequency components from each of the plurality of the electrophysiological frequency component sets; and
determining whether the clustering of the phase angles is greater than a clustering threshold.

10. The method of claim 9, further comprising:
determining whether a mean phase angle of the phase angles of the one or more frequency components from each of the plurality of electrophysiological frequency component sets is within an expected phase angle region that is correlated with phase angles which produce plausible morphologies associated with the target biological response.

11. The method of claim 1, wherein determining, based on the phase angles of the one or more frequency components from each of the plurality of electrophysiological frequency component sets, whether the plurality of electrophysiological traces represent the target biological response comprises:
clustering the phase angles of the one or more frequency components from each of the plurality of electrophysiological frequency component sets; and
using the clustering of the phase angles in a closed-loop control function.

12. (canceled)

13. The method of claim 11, wherein using the clustering of the phase angles in a closed-loop control function comprises:
using the clustering of the phase angles to control at least one of a current level of an implantable medical device or sampling performed by an implantable medical device.

14. (canceled)

15. The method of claim 1, wherein determining, based on the phase angles of the one or more frequency components from each of the plurality of electrophysiological frequency component sets, whether the plurality of electrophysiological traces represent the target biological response comprises:
determining, based on the phase angles of the one or more frequency components from each of the plurality of electrophysiological frequency component sets, whether the plurality of electrophysiological traces represent an electrically evoked compound action potential (ECAP) response.

16. The method of claim 1, wherein determining, based on the phase angles of the one or more frequency components from each of the plurality of electrophysiological frequency component sets, whether the plurality of electrophysiological traces represent the target biological response comprises:
determining, based on the phase angles of the one or more frequency components from each of the plurality of electrophysiological frequency component sets, whether the plurality of electrophysiological traces represent an electrocochleography (ECoG) response.

17. The method of claim 1, further comprising:
generating one or more outputs representative of the determination of whether the plurality of electrophysiological traces represent the target biological response.

18. (canceled)

19. The method of claim 1, further comprising:
generating one or more outputs indicating a parameter associated with the phase angles of the one or more frequency components from each of the plurality of electrophysiological frequency component sets.

20. The method of claim 19, wherein the parameter associated with the phase angles of the one or more frequency components comprises a concentration associated with a clustering of the phase angles of the one or more frequency components from each of the plurality of electrophysiological frequency component sets.

21. The method of claim 19, further comprising:
implementing a closed-loop control at an implantable medical device implantable in the recipient using the parameter associated with the phase angles of the one or more frequency components from each of the plurality of electrophysiological frequency component sets.

22. (canceled)

23. The method of claim 1, further comprising:
determining that a frequency component of at least one of the plurality of electrophysiological frequency component sets is an out-of-family frequency component; and
excluding the out-of-family frequency component of at least one of the plurality of electrophysiological frequency component sets from use in determining whether the plurality of electrophysiological traces represent a target biological response.

24. The method of claim 23, further comprising:
determining that the frequency component of at least one of the plurality of electrophysiological frequency component sets is an out-of-family frequency component based on an incidence of sound associated with the frequency component.

25. A system, comprising:
at least one electrode configured to be implanted in a recipient;
a recording module configured to record a plurality of sets of evoked electrophysiological signals from tissue of the recipient via the at least one electrode; and
one or more processors configured to:
generate a number of frequency component sets from the plurality of sets of evoked electrophysiological signals,
determine phase angles of one or more frequency components from each of a plurality of the frequency component sets, and
cluster the phase angles to determine whether the plurality of sets of evoked electrophysiological signals are associated with a predetermined evoked biological response.

26. The system of claim 25, further comprising a stimulator unit configured to deliver one or more electrical stimulation signals to the tissue of the recipient to evoke the electrophysiological signals.

27. The system of claim 25, further comprising a receiver configured to deliver one or more acoustical stimulation signals to the tissue of the recipient to evoke the electrophysiological signals.

28. The system of claim 25, further comprising an actuator configured to deliver one or more mechanical stimulation signals to the tissue of the recipient to evoke the electrophysiological signals.

29. The system of claim 25, wherein to record each of the plurality of sets of evoked electrophysiological signals, the recording module is configured to record a plurality of neuron action potentials for a period of time following delivery of a stimulation signal set to the tissue of the recipient.

30. The system of claim 25, wherein to generate the frequency component sets from the plurality of sets of evoked electrophysiological signals, the one or more processors are configured to:
extract phase and frequency components from each of the plurality of sets of evoked electrophysiological signals.

31. The system of claim 25, wherein to determine phase angles of one or more frequency components from each of the plurality of the frequency component sets, the one or more processors are configured to:
determine phase angles of a frequency component associated with a fundamental frequency of the predetermined evoked biological response.

32. The system of claim 25, wherein to cluster the phase angles to determine whether the plurality of sets of evoked electrophysiological signals are associated with a predetermined evoked biological response, the one or more processors are configured to:
determine whether the clustering of the phase angles is greater than a clustering threshold.

33. The system of claim 32, wherein the one or more processors are configured to:
determine whether a mean phase angle of the phase angles of the one or more frequency components from each of the plurality of the frequency component sets is within an expected phase angle region that is correlated with phase angles which produce plausible morphologies associated with the predetermined evoked biological response.

34. The system of claim 25, wherein to cluster the phase angles to determine whether the plurality of sets of evoked electrophysiological signals are associated with a predetermined evoked biological response, the one or more processors are configured to:
use the clustering of the phase angles in a closed-loop control function.

35. (canceled)

36. The system of claim 34, wherein to use the clustering of the phase angles in a closed-loop control function, the one or more processors are configured to:
use the clustering of the phase angles to control at least one of a current level of an implantable medical device or sampling performed by the implantable medical device.

37-56. (canceled)
